(19) United States
(12) Patent Application Publication
(10) Pub. No.: US 2006/0058626 A1
(43) Pub. Date: Mar. 16, 2006

(54) Universal healthcare communication systems and methods

(76) Inventors: Sanford B. Weiss, Los Angeles, CA (US); Ashok Dave, Sherman Oaks, CA (US)

Correspondence Address: STETINA BRUNDA GARRED & BRUCKER, 75 ENTERPRISE, SUITE 250, ALISO VIEJO, CA, 92656, US

(21) Appl. No.: 11/247,458

(22) Filed: Oct. 11, 2005

Related U.S. Application Data
(63) Continuation-in-part of application No. 11/139,828, filed on May 27, 2005.
Application No. 11/139,828 is a continuation-in-part of application No. 10/921,637, filed on Aug. 18, 2004.

(51) Int. Cl. A61B 5/05

(52) U.S. Cl. 600/407

(57) ABSTRACT

Medical communication systems and related methods are provided for facilitating the sending of medical treatment related communications to users of various user communication devices, such as wireless devices, over one or more networks. Users of such a communication device may securely log on to a host computer system and access information through the communication device or otherwise. Such information is accessible to all affected entities involved in the administration of healthcare, including physicians, hospital staff, healthcare administrators, skilled nursing/assisted living facilities, pharmacists and family members/guardians of patients. By utilizing various embodiments of the systems and methods, medical professionals and/or other users can receive timely updates of medical data related to a particular patient independent of location or time of day.

### CROSS-REFERENCE TO RELATED APPLICATIONS

This application is a continuation-in-part of, and claims the benefit of, U.S. patent application No. 11/139,828, filed on May 27, 2005, which is a continuation-in-part of, and claims the benefit of, U.S. patent application No. 10/921,637, filed on Aug. 18, 2004, each of which is incorporated by reference herein.

### STATEMENT RE: FEDERALLY SPONSORED RESEARCH/DEVELOPMENT

Not Applicable

### BACKGROUND OF THE INVENTION

1. Field of the Invention

The present invention is directed to technology for facilitating medical data alert notifications and communications between physicians, various healthcare providers, hospitals, outpatient facilities, healthcare institutions, insurance carriers, skilled nursing/assisted living facilities, patients, and their families/guardians.

2. Description of the Related Art

In recent years, improvements in communication technology have resulted in a wide array of communication options becoming available to large numbers of persons. In particular, the availability of mobile telephones, such as cellular phones and other wireless telephony devices, has improved the ability of users to keep in touch with each other, independent of location.

Typically, mobile phones are used for facilitating voice conversations and voicemail exchanges. Unfortunately, however, such devices are generally less useful for providing significant communication of other data. Mobile phones typically lack connectivity or functionality to link them to computing devices and/or databases that could support such data exchange in a convenient manner. Indeed, the communication of data between mobile phones is often cumbersome at best, and can be limited by differences in technology. For example, users of one type of communication technology, such as TDMA access technology, may be unable to exchange data with users of other types of communication technology, such as CDMA access technology.

These and other limitations can become particularly acute in the medical field. Like many other professionals, those in the medical field are often required to be reachable outside of their normal office hours. This is especially true in the case of medical emergencies, which occur at all times of day and night, and also frequently while medical professionals may be traveling or away from the office during after hours. In this regard, it can be extremely problematic to the extent an emergency room physician is unable to get in contact with a patient's primary care physician and/or family members/guardians/individuals possessing powers of attorney to make medical decisions on behalf of the patient. Along these lines, it is typically necessary for the emergency room physician to know of any pre-existing medical conditions, medications the patient may be taking, and other crucial information regarding the patient, such as blood type, allergies, and the like. Indeed, such situation occurs with great frequency for patients residing in nursing homes/skilled nursing facilities who typically are already compromised in terms of health and must make frequent trips between such nursing home/skilled nursing facility to hospitals where more immediate and comprehensive medical care can be rendered. Because many such patients are elderly and frequently suffer from dementia, either brought on by medications or medical conditions such as Alzheimer's, such patients are typically incapable of communicating with the treating physician to impart the necessary medical information, and much less so during an emergency.

To facilitate such communications while out of the office, medical professionals (i.e. doctors and other professionals in the field) typically rely on cellular phones and/or pagers to maintain communication with their offices, hospitals, nursing homes/assisted living facilities and the like. However, the use of such tools in combination with existing communication systems do not necessarily provide for the exchange of significant amounts of data as may be required to make meaningful decisions affecting treatment. Moreover, such communication systems are inoperative to provide a comprehensive network by which medical information can be exchanged not only between physicians, but also between other key entities involved in the administration of healthcare, such as information from labs regarding laboratory tests, nurses and other personnel responsible for overseeing the administration of healthcare, and perhaps most importantly, family members/guardians for the patient and/or other individuals having the necessary authority to make medical decisions oh behalf of the patient, who might possess key information about the patient as well as the patient's wishes involving crucial medical decisions, such as the use of heroic measures, organ donation, blood transfusion and the like. Many times, too, hospital and healthcare administrators must be able to contact physicians in order to obtain authorization necessary to treat/charge patients.

As such, these limitations can render it difficult for medical professionals to make decisions remotely that could affect patient care. Without sufficient communication tools, medical professionals can be forced to return to an office, hospital, and/or patient location in order to access medical information necessary for making informed decisions affecting patient care. These same limitations can also apply to other affected individuals, such as family members/guardians of the patient, who frequently need to be consulted and informed about the patient, as well as assist in determining an appropriate course of care. It will be appreciated that such limitations can be especially problematic when emergency situations arise and time is of the essence. Consequently, important and potentially life-saving decisions could be significantly delayed as a result of currently-available communication tools.

Existing communication systems also fail to provide medical information between various healthcare administration entities and medical professionals with data necessary to make a proper diagnosis or implement a particular treatment. For example, a doctor's treatment of a given patient may be dependant on the results of various tests being performed at another medical facility. As a result, patient treatment may be held in abeyance pending the doctor's receipt and review of the test results. In such cases, delays in receiving the patient test results can necessarily result in delays in patient treatment. Unfortunately, existing systems can require doctors to wait unacceptable periods of time before such information is eventually received by mail at the doctor's place of business. Moreover, even to the extent such information is available, further information, such as that provided by family members/guardians/those with the requisite power of attorney to make medical decisions on behalf of the patient, must further be taken into consideration but yet often times there is lacking any type of effective communication system by which crucial information can be obtained from such individuals.

### BRIEF SUMMARY OF THE INVENTION

The present disclosure, in various aspects, provides for various medical alert communication systems and related methods for providing alert notifications to remote user communication devices, such as wireless devices, over one or more networks. The present disclosure further provides for medical communication systems that enable virtually all types of entities involved in the administration of healthcare to a particular patient to possess means to communicate with one another to thus facilitate the exchange of medical information key to determine the status of and appropriate treatment for the patient, as well as facilitate the exchange of information necessary to provide an optimal degree of care to the patient in accordance with the patient's wishes in a dynamic and timely manner.

For example, a medical communications system can be provided comprising a server, a medical database in communication with the server, and an application running on the server for performing a method for providing medical data alert notifications. The method performed by the application can comprise a plurality of steps. Medical data associated with the database can be detected, and an alert notification can be generated in response to such detection. The alert notification can be implemented to identify the existence of the medical data being available from the system. The alert notification can then be broadcasted over a network to a user's communication device. A user request to access the medical data can be received by the system. In response, the medical data can be provided to the user's communication device. The user can then respond back via the system to other authorized users on the network.

In various embodiments, the network can be a wireless network and the user communication device can be a wireless device in communication with the system through the wireless network.

In other embodiments, the user communication device can be implemented as a personal digital assistant (PDA), a mobile telephone, and/or any wireless communication device.

Alert notifications contemplated by the system can be implemented in accordance with various ways, including voice-based alert notifications, text-based alert notifications, and/or other ways. The medical data provided to the user communication device can be formatted in a data format selected from the group consisting of: a digital image, digital video and/or real time visual monitoring, an audio file, and a text document.

A method of interacting with a medical communications system is contemplated by the present disclosure. In one embodiment, an alert notification can be received at a user's communication device. The alert notification can inform the user of the existence of medical data available from the system. The system can be accessed in response to the alert, and medical data can be received from the system. Such medical data can then be displayed on the user's communication device. The user's device can be used to respond back to other authorized entities on the network.

Along these lines, it is contemplated that such medical data can be shared not only between a particular facility/physician, but may also be disseminated and accessed by all those persons involved in or affected by the administration of healthcare to a particular patient. For example, it is expressly contemplated that the system will be operative to provide communications between healthcare personnel treating a particular patient at a first facility, such as a nursing home; hospital personnel, who may be involved in treating the patient at a hospital for an acute condition; the patient's primary care physician; labs and medical imaging facilities that may have and/or generate crucial information regarding the patient's condition; insurance carriers who are involved in the reimbursement for fees incurred as a result of the administration of healthcare; and family members/guardians for the patient who have a right to know about the patient's medical condition and the ability to make crucial healthcare decisions/provide additional healthcare information, as may be necessary or helpful.

These as well as other embodiments contemplated by the present disclosure will be more fully set forth in the detailed description below and the figures submitted herewith.

### BRIEF DESCRIPTION OF THE DRAWINGS

FIG. 1 illustrates a block diagram of a medical communications system for providing alert notifications over various networks to a plurality of user communication devices in accordance with an embodiment of the present invention.

FIG. 2 illustrates various technology components that can be provided as medical data sources in accordance with an embodiment of the present invention.

FIG. 3 illustrates a process for sending, receiving, and responding to an alert notification issued in accordance with an embodiment of the present invention.

FIG. 4 illustrates a schematic network of entities involved in the administration of healthcare and/or have information or are involved in the decision making process concerning the care and medical treatment of a particular patient, and how the medical communication system of the present invention is operative to facilitate communications therebetween.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

Medical alert communication systems and related methods can be provided for sending customized alert notifications to remote user communication devices, such as wireless devices, over one or more networks. Upon receiving an alert notification, a user of such a communication device may securely log on to a host computer system and access further information. Such information may include, for example, medical data (i.e. medical information) referenced by the alert notification.

Various embodiments of the system can provide support for selectively broadcasting voice-based messages, text-based messages, video-based images and/or other customized messages to wireless devices and/or devices in communication with the system through landlines. Further embodiments can provide support for browser-based access to medical data through the Internet and/or other computer networks. Although the system 100 and related methods are chiefly described herein in relation to medical data, it will be appreciated that the various embodiments of the system and related methods can be implemented to support other types of data, where appropriate. Along these lines, it is expressly contemplated that the system and related methods may be operative to simply serve as a direct communications link between all individuals and entities involved in the administration of healthcare to a particular patient, and expressly contemplates ongoing and continuous accessibility to patient monitoring and medical information concerning a particular patient by physicians, healthcare personnel, insurance carriers and family/guardians of the patient, among others intimately involved in the administration of healthcare.

It is contemplated that various embodiments of the system can be advantageously implemented to be device and/or network independent, permitting the broadcast of alert notifications to user communication devices in communication with a plurality of different types of networks, such as wireless networks. In such embodiments, the system can provide broad capabilities for sending alert notifications to various devices, independent of the particular device and/or network utilized by the device. For example, it is contemplated that a system of such an embodiment could provide for the broadcasting of customized alert notifications sent to every person in the United States utilizing an appropriate communication device as well as persons outside the United States utilizing global GSM-based communication devices, the communication devices having Internet access, and/or other appropriate communication devices and networks.

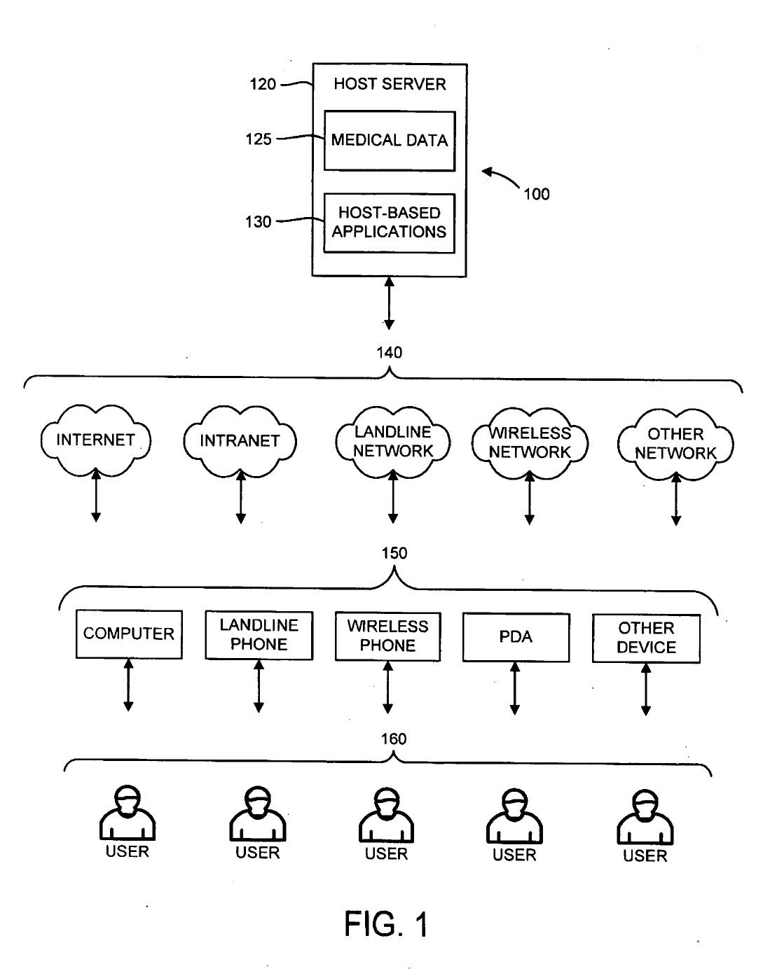

Turning to the figures of the present disclosure, FIG. 1 illustrates a block diagram of a medical communications system 100 for providing alert notifications over various networks 140 to a plurality of user communication devices 150 in accordance with an embodiment of the present invention. A host server 120 can be provided for facilitating the communication of alert notifications and medical data as further described herein. In one embodiment, server 120 can be implemented as a server supporting Microsoft Internet Information Services (IIS) 6.0 and Microsoft .Net v 1.1.

As illustrated, server 120 can be implemented to access various medical data associated with a medical database 125 of host server 120. However, it will be appreciated that medical database 125 can alternatively be implemented separate from, and in communication with, host server 120. In one embodiment, medical database 125 can be implemented on a server compatible with MS/SQL Server 2000.

One or more host-based applications 130 can be provided on server 120 for facilitating the processing features of system 100. In various embodiments, applications 130 can be implemented as compiled code running on ASP.NET files. VB.NET, and/or Thwate SSL 128-bit encryption certificate services can also be supported. Connection specifications can be embedded within the application code and associated configuration files. Host-based applications 130 can be implemented to utilize IIS 6.0 SSL features for supporting secured access. Accordingly, any user communication devices 160 that are not supporting secure sockets layer (SSL) at runtime (for example, web browsers running on computers or PDAs that do not comply and/or allow SSL web access) can be denied access to system 100.

Server 120 can be implemented to communicate with one or more networks 140. As set forth in FIG. 1, a plurality of different networks can be supported, including but not limited to: the Internet, intranets, landline networks, wireless networks, and/or other networks known in the art.

A plurality of user communication devices 150 can be provided in communication with the networks 140 supported by system 100. As illustrated, user communication devices 150 can include, but need not be limited to: computers, landline telephones, wireless telephones, person digital assistants (PDAs), and/or other mobile or non-mobile user devices known in the art for providing electronic communication. It will be appreciated that, where applicable, any of the user communication devices 150 can communicate over one or more of the networks 140 if the user communication device 150 supports compatibility with the network 140. In one embodiment, user communication devices 150 that are PDAs can be provided with a web browser compatible with SSL.

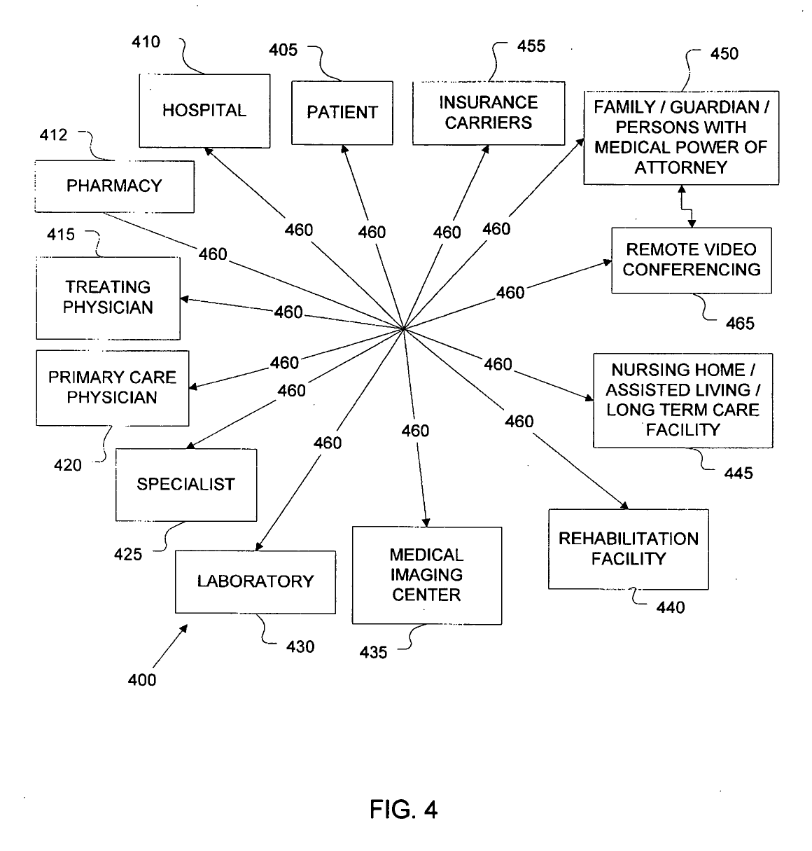

Each of user communication devices 150 can be utilized by one or more users 160. It will be appreciated that in embodiments where system 100 is deployed in the medical field, users 160 can be medical professionals, such as doctors and/or other persons with whom the sharing of medical data may be desired. It is further contemplated that other entities involved in the administration of healthcare may further have access to the medical data, and can include insurance carriers, labs, medical imaging facilities, rehabilitation facilities, nursing homes/assisted living facilities, in-home care providers and, where appropriate, relatives/guardians of the patient as well as those individuals possessing power of attorney to make medical decisions on behalf of the patient, as discussed in further detail below in connection with FIG. 4.

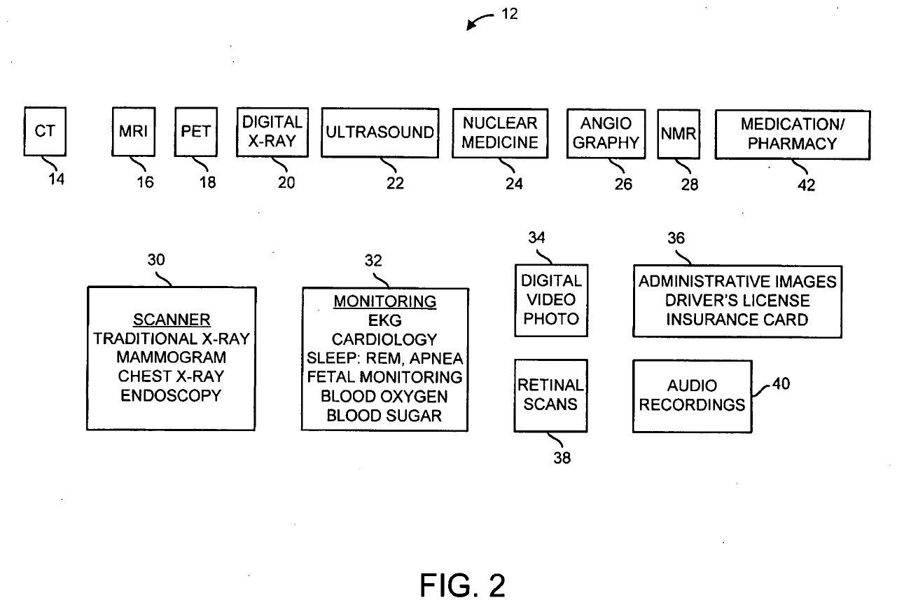

Referring now to FIG. 2, there is shown various media modalities 12 which can be provided as medical data sources for medical data associated with medical database 125. As illustrated, such modalities may include, but need not be limited to computerized tomography (CT) 14, magnetic resonance imaging (MRI) 16, positron emission tomography (PET) 18, digital X-ray 20, ultrasound 22, nuclear medicine 24, angiography 26, and nuclear magnetic resonance (NMR) 28. Other non-digital images 30 can be converted into digital form through the use of a film digitizer or scanner 30. These images may include more traditional X-ray radiography such as chest X-rays or mammograms, or images taken through endoscopes. The output from physiological monitoring systems 32 such as wave patterns recorded in cardiology EKGs, sleep clinic REM or sleep apnea measurements, or in fetal monitoring can also be medical data sources. Images, movies, and sound may be recorded from any device 34, including but not limited to digital cameras, camcorders, camera cell phones, and the like. Photocopies or scanned images 36 of driver's licenses, social security cards, and other identification, as well as virtually any document can also be digitized through the scanner. Retinal scans 38, fingerprint data, and audio recordings 40 also may be included. Additionally, medication and other pharmaceutical data 42, such as the drug allergies, may be included. In this respect, it is expressly contemplated that all of the types of medical information may be assimilated according to the teachings of Applicants' pending U.S. patent application No. 10/921,637 and U.S. patent application No. 11/139,828, entitled “Medical alert communication systems and methods” filed May 27, 2005, the teachings of which are expressly incorporated herein by reference.

As further described herein, the various medical data sources illustrated in FIG. 2 can be provided in medical database 125 in accordance with data formats compatible with one or more of the user communication devices 150. For example, the medical data can be formatted in a data format selected from the group consisting of: a digital image, an audio file, a text document, and/or other appropriate data formats.

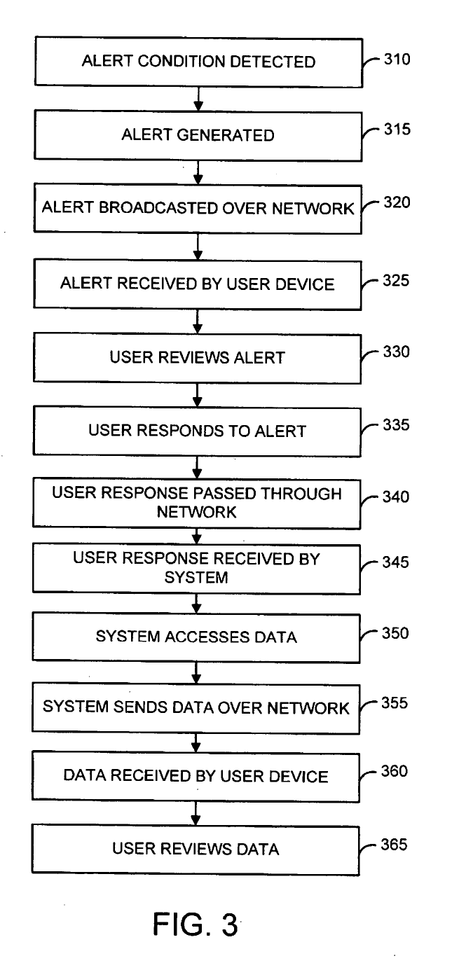

FIG. 3 illustrates a process for sending, receiving, and responding to an alert notification issued in accordance with an embodiment of the present invention. At step 310, a host-based application 130 detects a condition giving rise to an alert notification. Such an alert condition can be any condition detectable by one or more of the host-based applications running on host server 120. For example, in the case of medical data, a host-based application 130 may detect the presence of new and/or updated medical data associated with medical database 125, whether stored therein or otherwise. It will be appreciated that such new/updated medical data can be received by host server 120 through communication with one or more of networks 140, and passed to medical database 125.

Upon detection of an alert condition in step 310, a host-based application 130 can generate an alert notification (step 315). Such an alert notification can comprise an appropriately-formatted communication capable of being received by and accessed on one or more of the user communication devices 150. For example, text-based alert notifications, voice-based alert notifications, and alert notifications in other appropriate data formats are contemplated. In addition, the alert notifications can be directed to particular user communication devices 150 if desired, thereby permitting alert notifications to be selectively directed to, and customized for, particular users 160 of the user communication devices 150.

As discussed, the alert notification generated in step 315 can comprise a notification that new and/or updated medical data has become associated with medical database 125. Such data may include, but need not be limited to, newly received test results, a change in a patient's medical condition, and/or other medical data or related information.

At step 320, the alert notification generated in step 315 can be broadcasted across one or more of the networks 140. It will be appreciated that in order to support a plurality of different data formats and communications standards supported and/or required by various networks 140, the alert notification can be converted by one or more of the host-based applications 130 into an appropriately-formatted communication prior to the broadcasting of step 320.

Following the broadcast of the alert notification, it can be received by one or more of the user communication devices 150 (step 325) through one or more of the networks 140. It will be appreciated that various user communication devices 150 may support a variety of features which may be employed to notify the user 160 that an alert notification has been received. For example, the user communication device 150 may exhibit an audible sound, vibration, visual display, and/or another appropriate indication to signify that an alert notification has been received.

At step 330, the user 160 can review the alert notification received by the user communication device 150 in step 325. It will be appreciated that such user review of the alert notification can be performed in response to the user's perception of an indication provided by the user communication device 150 to signify that an alert notification has been received.

The user 160 may then choose to respond to the alert notification in step 335. For example, if the user 160 desires to access the system 100 to review the updated medical data available from the system 100 that was the subject of the alert notification, the user 160 may initiate a request from the user communication device 150 to access the medical data. In various embodiments, such a request may include logging in the user 160 to the system 100 through an authentication/authorization process, and sending a request to access the medical data.

At step 340, the user response of step 335 is passed from the user's communication device 150 through an appropriate one or more of networks 140 compatible with the user's communication device 150. The user response can then be received by server 120 of system 100 from one or more of the networks 140 (step 345).

It is contemplated that the user response of step 335 can be sent from the same user communication device 150 that received the alert notification in step 325. However, it is also contemplated that one or more alternative user communication devices 150 may be used to send the user response and support the further interaction between the user 160 and system 100. For example, if a user 160 receives an alert notification on a particular wireless phone (step 325), it may be desirable for the user to access the medical data referenced by the alert through a PDA device which may exhibit a larger screen, thereby permitting the user to more easily view the medical data to be accessed from system 100. Similarly, the user may desire to receive alert notifications wirelessly (i.e. through a wireless user communication device 160), but access medical data through a landline-based user communication device 160.

Upon receiving the user request in step 345, one or more of the host-based applications 130 of system 100 accesses the medical data available from medical database 125. In this regard, it will be appreciated that the user response initiated by the user 160 in step 335 can advantageously reference the alert notification previously generated and broadcasted by the system in steps 315 and 320, respectively. As such, the alert notification can be implemented to reference the updated/new medical data available from medical database 125. Accordingly, by referencing the alert notification in the user response of step 335, the system 100 can be informed as to which medical data should be accessed in response to the user request.

In step 350, the system 100 accesses the medical data referenced by the alert notification in response to the user response of step 335. During step 350, a host-based application 130 can dynamically extract the particular medical data sought by the user response from medical database 125.

Following the accessing step 350, the system 100 can send the accessed data over one or more appropriate networks 150 to one or more user communication devices 160 (step 355). For example, the user communication device 160 to which the accessed data is sent can be the same user communication device 150 that initiated the user response in step 335. However, it will be appreciated that system 100 may be appropriately configured to send the accessed data to another user communication device 150 in the alternative and/or in addition to the original user communication device 150.

At step 360, the medical data accessed in step 350 and sent in step 355 can be received by a user communication device 150 and displayed, printed, played, and/or otherwise accessed thereon. As a result, the user 160 may review the medical data directly from the user communication device 160 (step 365) and choose to take appropriate action in response to the medical data.

Advantageously, in some embodiments, the medical data accessed on user communication device 160 can be stored in only volatile memory of the user communication device 160, and only while the user communication device 160 maintains a communication link with system 100. As such, it will be appreciated that by not storing the medical data in semi-permanent and/or permanent memory of the user communication device 160, the likelihood of inadvertent disclosure of private/personal medical data can be reduced.

In another aspect of system 100, alert notifications, user responses, and/or medical data accessed by the system 100 can be logged for security and audit purposes, and for compliance with HIPPA standards for healthcare deployment.

System 100 can also be implemented to limit user 160 interaction with the system 100 to pre-selected time intervals (for example, 5 minute sessions or 10 minute sessions). Upon the expiration of a time interval, the user 160 may be required to re-login (for example, through step 335) in order to further access features of the system 100.

It will be appreciated that through the execution of the process of FIG. 3, real-time alert notifications can be provided to users 160 of various user communication devices 150. Users 160 can then respond to such alert notifications and access medical data referenced by the alert notifications as desired through one or more user communication devices 150 over one or more networks 140 compatible with the user communication devices 150. As a result, medical professionals and/or other users 160 can receive timely updates of meaningfully significant amounts of medical data independent of location or time of day.

Referring now to FIG. 4, there is shown a communications web 400 which identifies several entities involved in the administration of healthcare to a patient 405 and how the system and methods of the present invention are operative to provide communications links 460 to one another to thus ensure as comprehensive a framework as is possible to allow for the exchange of medical data concerning healthcare administered to a patient 405. Such arrangement is specifically configured to attempt to ensure that an optimal degree of care can not only be administered, but administered as efficiently as possible and in accordance with the patient's desires.

As illustrated, the various entities that may be involved in the administration of healthcare may include a particular hospital 410 where the patient 405 is being treated and the treating physician 415 who may be responsible for actually administering treatment to the patient 405. Along these lines, it is contemplated that the treating physician 415 may be an emergency room physician responsible for administering immediate healthcare in response to a particular emergency. Further included within such communications web is the patient's primary care physician 420, as well as one or more specialists 425 that may be needed for consultation to treat the patient 405 for a particular condition, disease, injury or the like. The inclusion of pharmacists 412 for providing a variety of medication related consultation to patient 405, as well as treating physician 415, primary care physician 420, and specialist 425 is also contemplated.

Moreover, communications web 400 may include other entities responsible for the caring of the patient 405, as well as retrieving, generating and conveying key medical information related to the patient 405 and/or a particular condition of the patient 405. As illustrated, laboratory 430 may be integrated within the communications web 400 to thus provide information regarding lab tests, such as blood tests and the like, essential for making proper diagnosis and patient evaluation. Similarly, there is included medical imaging center 435 and rehabilitation facility 440, each of which may be involved in providing patient care and/or providing information related to the patient's condition. Along these lines, it is frequently necessary for all healthcare administration personnel, and in particular, treating physicians 415, primary care physicians 420 and, where applicable, the relevant specialists 425 to have access to information provided by such entities 430, 435, 440 as quickly as possible. Still further, it is contemplated that entities responsible for providing long-term care, such as nursing homes/assisted living and other long term care facilities 445 may be included insofar as patients can and frequently will comprise disabled/elderly individuals who must rely upon healthcare to be administered at a nursing home/assisted living and other long term care facility 445. Indeed, it is contemplated that the systems and methods of the present invention will be particularly well suited for relaying rapid and accurate medical information to and from physicians and other healthcare providers for patients that reside in such facilities 445. Along these lines, it is contemplated that given the growing demographic of individuals residing in such nursing home/assisted living and other long term care facilities 445, it is expressly contemplated that the systems and methods of the present invention will play an integral part of providing optimal and cost-effective healthcare on-site at such facilities 445, and thus substantially minimizing the need to duplicate and forward patient records and decrease unnecessary patient visits to hospitals 410 and other acute care facilities.

A still further aspect of the invention is the inclusion of those individuals and activities who are related and/or responsible for making medical decisions related to the patient 405, and paying/authorizing treatment, such as insurance carriers 455. Such individuals 450 will typically include family members, guardians, and persons with medical power of attorney who must render decisions on behalf of the patient 405. In this respect, it is contemplated that, such group of individuals 450 may be readily consulted to the extent medical decisions must be made regarding a particular type of treatment and/or care. Exemplary of such decisions include whether or not to utilize heroic measures in resuscitating the patient 405, such as the use of a ventilator; whether or not the patient 405 wishes to donate organs, and other medical information that may not be readily known about the patient 405, such as whether or not the patient has any particular allergies, family history of disease, is on any type of medication, or has any type of pre-existing condition not readily known. Similar input may be sought from insurance carriers 455 who often times must necessarily be consulted before treatment is authorized. Accordingly, where applicable, it is contemplated that all medical alert information referenced herein may further be directed to such individuals 450, as well as all other entities identified in the communications web 400.

In yet a further aspect of the present invention there is provided means for not only relaying medical data, but also overseeing the basic administration of care to a patient 405. To that end, it is contemplated that any of the communications links 460 may be readily accessed at any time by anyone within the communications web 400 to determine whether of not the optimal degree of care is being administered to the patient 405. Along these lines, it is expressly contemplated that the link between family members/guardians, and the like 450 to nursing homes/assisted living and other long term care facilities 445 may be easily and readily utilized to enable family members 450 and the like to stay in touch with patients 405 confined to nursing homes/assisted living and other long term care facilities 445.

In fact, it is expressly contemplated that such communications link provided between family/guardians, and the like 450 and nursing homes/assisted living and other long term care facilities 445 may enable such family members 450 with means to visit the patient 405 that may be confined to such facilities 445. For example, it is contemplated that the telecommunications equipment and systems referenced above can be utilized to provide web-based video and audio data to thus enable family members 450 to readily communicate with patients 405 in nursing homes/assisted living and other long term care facilities 445.

Such web-based video and audio data is typically provided by remote video conferencing 465, and serves as an interface between communications web 400 and the family members 450. Remote video conferencing 465 may be provided by any one of means recognized in the art for the real-time visual monitoring of remote locations, and may be accessed from the personal computer of family members 450, or from any computer system capable of accessing communications web 400 such as office computers or internet caf\acute{e} computers, which are provided by way of example only and not of limitation. Furthermore, remote video conferencing 465 may be accessed by anyone having authorization from family members 450 that can use the same.

As will be readily appreciated by those skilled in the art, real-time visual monitoring of remote locations is well known. In particular, digital video cameras are connected to personal computers, and the captured footage is processed and transported via a network medium to a remote computer, where it is viewed by the user. In such systems, the digital video camera is typically comprised of an optical lens, a Charge Coupled Device (CCD) sensor, and a local data transfer means.

As is well understood, a CCD sensor is an integrated circuit containing an array of photodiodes coupled with capacitors, with each such point defining a pixel. When light strikes the photodiode after focusing of the same by the optical lens, electrons are freed and accumulate in the capacitors. In order to obtain color information, a Bayer mask is applied over the CCD. By way of an analog-to-digital converter, charge values at each pixel can be digitized, and converted to a data stream of varying red, green, blue (RGB) values. This process is repeated for each row of pixels, until the entire frame has been processed. For video signals, this process is continued indefinitely. The stream of data is then typically transferred to a personal computer via an I.E.E.E. 1394a or USB 1.2/2.0 connection.

Upon receipt of raw RGB data at the computer, a software program known as a codec encodes the data for transmission across a computer network. The video codec performs a transformation on the data that compress the signal so that it is better suited for transmission across limited bandwidth networks. Among the video codecs well known in the art include MPEG-1 (VCD), MPEG-2 (DVD), MPEG-4, and H.264. Audio data often accompanies video data, and so audio data is similarly encoded. As a person of ordinary skill in the art will recognize, audio codecs include MPEG-1 Audio Layer 3, also known as MP3, Windows Media Audio (WMA), and Advanced Audio Coding (AAC). In order to transport both audio and video data in a synchronized fashion, the encoded audio data and the encoded video data is encapsulated into a video file container, such as .mp4, .avi, or .mov.

A person of ordinary skill in the art will recognize that a variety of transport means can be used to transmit the encapsulated video and audio data to a remote network location. However, the most common means is via a datagram protocol, for example, the User Datagram Protocol (UDP). The data is deconstructed into a series of small packets, called datagrams, and sent to the remote computer.

Upon receipt at the remote computer, the packets are reconstructed, and the resultant data is de-encapsulated into separate audio and video data. The separate audio and video data is then decoded by the codec, and sent to one or more output devices, namely loudspeakers and a graphical display, respectively.

Where applicable, the present invention can be implemented using hardware, software, and/or combinations of hardware and software. Also where applicable, the various hardware components and/or software components set forth herein can be combined into composite components comprising software, hardware, and/or both without departing from the spirit of the present invention. Where applicable, the various hardware components and/or software components set forth herein can be dissected into sub-components comprising software, hardware, and/or both without departing from the spirit of the present invention. In addition, where applicable, it is contemplated that software components can be implemented as hardware components, and vice-versa.

Software in accordance with the present invention, such as program code and/or data, can be stored on one or more computer readable mediums. It is also contemplated that software identified herein can be implemented using one or more general purpose computers, specific purpose computers, and/or computer systems, networked and/or otherwise.

Where applicable, the ordering of various steps described herein can be changed, combined into composite steps, and/or dissected into sub-steps to provide the functionality described herein.

The foregoing disclosure is not intended to limit the present invention to the precise forms or particular fields of use disclosed. It is contemplated that various alternate embodiments and/or modifications to the present invention, whether explicitly described or implied herein, are possible in light of the disclosure.

What is claimed is:

1. A medical communications system comprising:
a server;
a medical database in communication with the server; said database being accessible by individual users selected from the group consisting of physicians, nurses, pharmacists, healthcare administrators, insurance carriers, healthcare providers and family members/guardians of patients associated with the administration of care and medical treatment to a patient; and
an application for performing a method for providing medical data alert notifications associated with the care and medical treatment of said patient, the application running on the server, the method comprising:
detecting medical data associated with the database, generating an alert notification in response to the detecting of medical data, the alert notification identifying the existence of the medical data being available from the system,
broadcasting the alert notification over a network to a user's communication device,
receiving a user request to access the medical data,
providing the medical data to the user's communication device in response to the user request, and
providing the capability for the user to respond back to the sending application and/or to other authorized users on the network.

2. The system of claim 1, wherein the network is a wireless network, the communication device is a wireless device in communication with the system through the wireless network.

3. The system of claim 2, wherein the communication device is a personal digital assistant (PDA).

4. The system of claim 2, wherein the communication device is a mobile telephone.

5. The system of claim 1, wherein he alert notification is a voice-based alert notification.

6. The system of claim 1, wherein the alert notification is a text-based alert notification.

7. The system of claim 1, wherein the medical data is formatted in a data format selected from the group consisting of:
a digital image;
an audio file; and
a text document.

8. A method for providing medical data amongst a variety of entities responsible for the administration of healthcare to a patient, the method comprising:
detecting medical data related to the patient associated with a database;
generating the response to the detecting of medical data, the data identifying the existence of the medical data being available from a medical communications system;
broadcasting the medical data over a network to a user's communication device, said user being selected from the group consisting of a hospital, a treating physician, a primary care physician, a specialist physician, a pharmacist, a laboratory, a medical imaging center, a rehabilitation facility, a nursing home/assisted living facility, a family member/guardian of said patient, and an insurance carrier;
receiving a user request to access the medical data; and
providing the medical data to the user's communication device in response to the user request.

9. The method of claim 8, wherein the network is a wireless network, the communication device is a wireless device in communication with the system through the wireless network.

10. The method of claim 9, wherein the communication device is a personal digital assistant (PDA).

11. The method of claim 9, wherein the communication device is a mobile telephone.

12. The method of claim 8, wherein the alert notification is a voice-based alert notification.

13. The method of claim 8, wherein the alert notification is a text-based alert notification.

14. The method of claim 8, wherein the medical data is formatted in a data format selected from the group consisting of:
a digital image;
a digital video;
an audio file; and
a text document.
